(19) United States
(12) Patent Application Publication
(10) Pub. No.: US 2002/0016262 A1
(43) Pub. Date: Feb. 7, 2002

(54) Pyrazole derivatives and herbicides containing the same

(75) Inventors: Kazufumi Nakamura, Sodegaura-shi (JP); Kazuyoshi Koike, Sodegaura-shi (JP); Masashi Sakamoto, Sodegaura-shi (JP); Ichiro Nasuno, Sodegaura-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(73) Assignee: IDEMITSU KOSAN CO., LTD., Tokyo (JP)

(21) Appl. No.: 09/910,049

(22) Filed: Jul. 23, 2001

Related U.S. Application Data
(63) Continuation of application No. 09/675,144, filed on Sep. 29, 2000, now abandoned.
Application No. 09/675,144 is a continuation of application No. 09/531,335, filed on Mar. 20, 2000, now abandoned.
Application No. 09/531,335 is a continuation of application No. 09/207,111, filed on Dec. 8, 1998, now abandoned.
Application No. 09/207,111 is a continuation of application No. 09/035,303, filed on Mar. 2, 1998, now abandoned.

(51) Int. Cl. A01N 43/56; C 07D 4 9/02

(52) U.S. Cl. 504/282; 548/364.4

(57) ABSTRACT

Disclosed are novel pyrazole derivatives which are free of phytotoxicity to corn and can control a broad range of cropland weeds at a low dosage and herbicides containing them, and the present invention provides pyrazole derivatives of the general formula (I), or salts thereof,

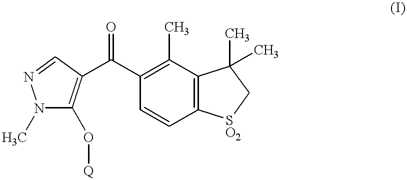

wherein Q is H, —SO₂—R¹, —CO—R¹ or —CH₂CO—R¹, in which R¹ is a C₁-C₈ alkyl group, a C₃-C₈ cycloalkyl group, a C₁-C₈ haloalkyl group or a phenyl group which may have a specific substituent, and herbicides containing these pyrazole derivatives or salts thereof as active ingredients.

### BACKGROUND OF THE INVENTION

1. Field of the Invention

The present invention relates to novel pyrazole derivatives and herbicides containing them. More specifically, it relates to pyrazole derivatives which can control a broad range of cropland weeds at a low dosage without causing phytotoxicity on corn, and herbicide containing them.

2. Description of Related Art

Herbicides are very important chemicals for labor-saving of weed control working and production improvement in horticultural crops. Herbicides have been therefore aggressively studied and developed for a long time, and a variety of chemicals are now practically used. However, it is still desired to develop novel herbicides having further superior herbicidal properties, particularly herbicides which can selectively control object weeds alone at a low dosage without causing phytotoxicity on cultivated crops.

During the planting time of corn, triazine-containing herbicides such as atrazine and acid anilide-containing herbicides such as alachlor and metolachlor have been conventionally used. However, atrazine shows low efficacy to grass weeds, and on the other hand, alachlor and metolachlor show low efficacy to broad-leaved weeds. It is therefore difficult at present to control grass weeds and broad-leaved weeds together simultaneously with a single herbicide. Further, these herbicides are undesirable in view of an environmental problem due to their high dosage requirement.

Meanwhile, it is known that specific 4-benzoyl derivatives have herbicidal activity (JP-A-63-122672, JP-A-63-122673, JP-A-63-170365, JP-A-1-52759, JP-A-2-173 and JP-A-2-288866). At present, pyrazolate having the following formula is commercially available as a herbicide.

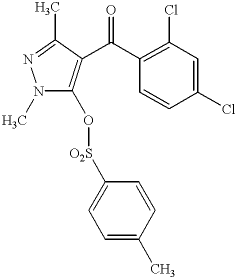

The above 4-benzoyl derivatives have herbicidal activity. However, they are insufficient for practical use, and are extremely poor in herbicidal activity, particularly, against grass weeds such as barnyardgrass and green foxtail.

There has been filed a patent application directed to a compound in which a benzo[b]thiophene ring and a pyrazole ring are combined for overcoming the above defects (WO96/25412).

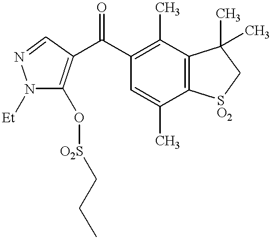

Further, WO97/08164 discloses the following compound.

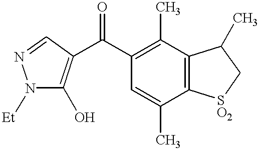

Benzo[b]thiophene derivatives including the above two compounds, specifically disclosed as examples in the above International Laid-open Publications, have higher herbicidal activity than the above benzoyl derivatives, while compounds having far higher herbicidal activity have been desired.

### DISCLOSURE OF THE INVENTION

Under the circumstances, it is a first object of the present invention to provide a novel pyrazole derivative which can control a broad range of upland soil weeds at a low dosage without causing phytotoxicity on corn.

It is another object of the present invention to provide a herbicide containing the above pyrazole derivative.

The present inventors have therefore made diligent studies for achieving the above objects, and as a result, have found that pyrazole derivatives having a specific structure formed by combining a benzo[b]thiophene ring and a pyrazole ring shows greatly improved activity against redroot pigweed (Amaranthus retroflexus) and weeds in its category and shows improved activity in soil treatment and further that it is free of phytotoxicity to corn. The present invention has been completed on the basis of the above finding.

That is, the first object of the present invention is achieved by pyrazole derivatives of the general formula (I), or salt thereof,

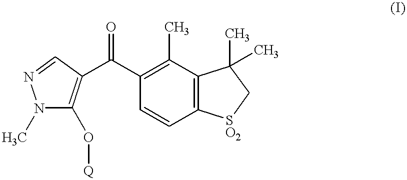

wherein Q is a hydrogen atom, a group of —SO₂—R¹, —CO—R¹ or —CH₂CO—R¹, in which R¹ is a C₁-C₈ alkyl group, a C₃-C₈ cycloalkyl group, a C₁-C₈ haloalkyl group or a group of the formula (II),

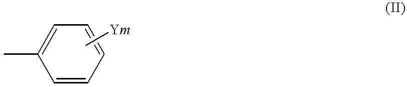

in which Y is a halogen atom, a nitro group, a C₁-C₄ alkyl group, a C₁-C₄ alkoxy group or a C₁-C₄ haloalkyl group, and m is an integer of 0 to 3, provided that when m is 2 or 3, each of Ys may be different or the same.

Further, the second object of the present invention is achieved by a herbicide containing, as an active ingredient, at least one selected from pyrazole derivatives of the above general formula (I) or salts thereof.

### BEST MODE OF THE INVENTION

The pyrazole derivative of the present invention will be first explained.

The pyrazole derivative of the present invention has the following general formula (I).

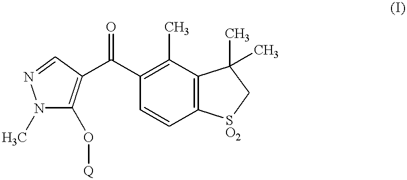

In the general formula (I), Q is a hydrogen atom or a group of —SO₂—R¹, —CO—R¹ or —CH₂CO—R¹, in which R¹ is a C₁-C₈ alkyl group, a C₃-C₈ cycloalkyl group, a C₁-C₈ haloalkyl group or a group of the formula (II).

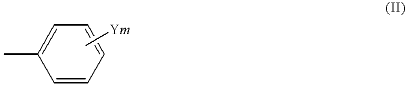

Examples of the C₁-C₈ alkyl group in the definition of R¹ include methyl, ethyl, propyl, butyl, pentyl, hexyl, heptyl and octyl, and of these, groups having at least 3 carbon atoms may be linear or branched. Examples of the C₃-C₈ cycloalkyl group in the definition of R¹ include cyclopropyl, cyclobutyl, cyclopentyl, cyclohexyl, cycloheptyl and cyclooctyl. Additionally, the C₃-C₈ cycloalkyl group may have a proper alkyl group whose total carbon atom number is 1 to 4 introduced on its ring. Examples of the C₁-C₈ haloalkyl group in the definition of R¹ include those obtained by replacing at least one hydrogen atoms of methyl, ethyl, propyl, butyl, pentyl, hexyl, heptyl or octyl with at least one halogen atoms such as fluorine, chlorine, bromine or iodine atom. Of these, C₁-C₈ haloalkyl groups having at least 3 carbon atoms may be linear or branched. Further, when at least two halogen atoms are substituted, the halogen atoms may be the same or different.

In the group of the above formula (II) in the definition of R¹, Y is a halogen atom (fluorine, chlorine, bromine or iodine), a nitro group, a C₁-C₄ alkyl group, a C₁-C₄ alkoxy group or a C₁-C₄ haloalkyl group. Examples of the C₁-C₄ alkyl group include methyl, ethyl, propyl and butyl. Of these, alkyl groups having 3 and 4 carbon atoms may be linear or branched. Examples of the C₁-C₄ alkoxy group include methoxy, ethoxy, propoxy and butoxy. Of these, alkoxy groups having 3 and 4 carbon atoms may be linear or branched. Further, examples of the C₁-C₄ haloalkyl group include those obtained by replacing at least one hydrogen atoms of methyl, ethycl, propyl or butyl with at least one halogen atoms such as fluorine, chlorine, bromine or iodine atom. Of these, haloalkyl groups having 3 and 4 carbon atoms may be linear or branched. When at least two halogen atoms are substituted, the halogen atoms may be the same or different. m is an integer of 0 to 3, and when m is 2 or 3, each of Ys may be the same or different.

The pyrazole derivative of the general formula (I) in which Q is a hydrogen atom, i.e., a compound of the general formula (Ia),

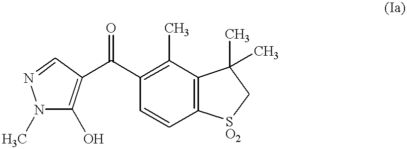

can have the following three structures of tautomerism, and the pyrazole derivative of the present invention includes all of these compounds.

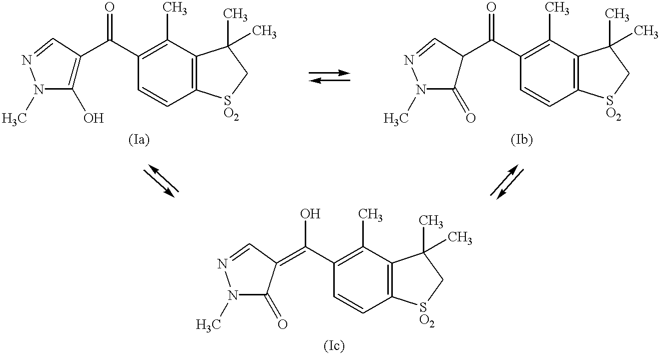

Further, the pyrazole derivative of the formula (Ia) is an acidic substance, and can be easily converted to salt by treating it with a base. This salt is also included in the pyrazole derivative of the present invention. The above base is not specially limited, and can be selected from known bases. The base includes organic bases such as amines and anilines and inorganic bases such as sodium compounds and potassium compounds. Examples of the amines include monoalkylamine, dialkylamine and trialkylamine. Alkyl groups of the alkylamines are generally C₁-C₄ alkyl groups. Examples of the anilines include aniline, monoalkylaniline and dialkylaniline. Alkyl groups of the alkylanilines are generally C₁-C₄ alkyl groups. Examples of the sodium compounds include sodium hydroxide and sodium carbonate. Examples of the potassium compounds include potassium hydroxide and potassium carbonate.

The herbicide of the present invention contains, as an active ingredient, at least one selected from pyrazole derivatives of the general formula (I) and the salts thereof, provided by the present invention. These compounds are used by mixing them with a liquid carrier such as a solvent or a solid carrier such as a mineral fine powder and preparing the resultant mixtures in the form of a wettable powder, an emulsifiable concentrate, a dust or granules. These compounds can be imparted with emulsifiability, dispersibility or spreadability by adding a surfactant when the above preparations are formed.

When the herbicide of the present invention is used in the form of a wettable powder, generally, 10 to 55% by weight of the pyrazole derivative or the salt thereof, provided by the present invention, 40 to 88% by weight of a solid carrier and 2 to 5% by weight of a surfactant are mixed to prepare a composition, and the composition can be used. When the herbicide of the present invention is used in the form of an emulsifiable concentrate, generally, it is sufficient to prepare a composition by mixing 20 to 50% by weight of the pyrazole derivative or the salt thereof, provided by the present invention, 35 to 75% by weight of a solvent and 5 to 15% by weight of a surfactant. When the herbicide of the present invention is used in the form of a dust, generally, it is sufficient to prepare a composition by mixing 1 to 15% by weight of the pyrazole derivative or the salt thereof, provided by the present invention, 80 to 97% by weight of a solid carrier and 2 to 5% by weight of a surfactant. Further, when the herbicide of the present invention is used in the form of granules, generally, it is sufficient to prepare a composition by mixing 1 to 15% by weight of the pyrazole derivative or the salt thereof, provided by the present invention, 80 to 97% by weight of a sold carrier and 2 to 5% by weight of a surfactant.

The above solid carrier is selected from fine mineral powders, and examples of the mineral fine powders include oxides such as diatomaceous earth and slaked lime, phosphates such as apatite, sulfates such as gypsum, and silicates such as talc, pyroferrite, clay, kaolin, bentonite, acid clay, white carbon, powdered quartz and powdered silica.

The solvent is selected from organic solvents. Specific examples of the solvent include aromatic hydrocarbons such as benzene, toluene and xylene, chlorinated hydrocarbons such as o-chlorotoluene, trichloroethane and trichloroethylene, alcohols such as cyclohexanol, amyl alcohol and ethylene glycol, ketones such as isophorone, cyclohexanone and cyclohexenyl-cyclohexanone, ethers such as butyl cellosolve, diethyl ether and methyl ethyl ether, esters such as isopropyl acetate, benzyl acetate and methyl phthalate, amides such as dimethylformamide, and mixtures of these.

Further, the surfactant can be selected from anionic surfactants (fatty acid salt, alkyl sulfate, alkylbenzenesulfonate, dialkylbenzenesuccinate alkyl phosphate, salt of naphthalenesulfonic acid formalin condensate and polyoxyethylenealkylsulfate), nonionic surfactants (polyoxyethylene alkyl ether, polyoxyethylene alkylphenol ether, polyoxyethylene alkyl ester, polyoxyethylenealkylamine, sorbitan fatty acid ester and polyoxyethylene sorbitan fatty acid ester), cationic surfactants and amphoteric surfactants (amino acid and betaine).

The herbicide of the present invention may contain, as an active ingredient, other herbicidally active component as required in combination with the pyrazole derivative of the general formula (I) or its salt. The “other” herbicidally active component includes known herbicides such as phenoxy-, diphenyl ether-, triazine-, urea-, carbamate-, thiocarbamate-, acid anilide-, pyrazole-, phosphoric acid-, sulfonylurea- and oxadiazone-based herbicides, and it can be properly selected from these herbicides as required.

Further, the herbicide of the present invention may be used as a mixture with any one of insecticides, bactericides, plant growth regulators and fertilizers.

The herbicide of the present invention can be used as a herbicide for upland soil by any method of pre-emergence treatment, pre-plant incorporation treatment and post-emergence treatment. The cropland weeds to which the compound of the present invention is applied include broad-leaved weeds such as solanaceous weeds typified by black nightshade (Solanum nigrum) and Jimsonweed (Datura stramonium); malvaceous weeds typified by velvetleaf (Abutilon theophrasti) and pricky sida (Sida spinosa); convolvulaceous weeds typified by morning-glories (Ipomoea spps.) such as tall morning-glory (Ipomoea purpurea) and hedge bindweeds (Calystegia spps.); amaranthaceous weeds typified by livid amaranth (Amaranthus lividus); composite weeds typified by cocklebur (Xanthium strumarium), common ragweed (Ambrosia artemisilfolia), sunflower (Helianthus annus), hairy galinsoga (Galinsoga ciliata), Canada thistle (Cirsium arvense), groundsel (Senecio vulgaris) and annual fleabane (Erigeron annus); cruciferous weeds typified by yellow cress (Rorippa indica), wild mustard (Sinapis arvensis) and shepherdspurse (Capsella bursa-pastris); polygonaceous weeds typified by smartweed (Polygonum blumei) and wild buckwheat (Polygonum convolvulus); portulacaceous weeds typified by common purslane (Portulaca oleracea); chenopodiaceous weeds typified by common lambsquaters (Chenopodium album), fig-leaved goosefoot (Chenopodium ficifolium) and kochia (Kochia scoparia); caryophyllaceous weeds typified by common chickweed (Stellaria media); scrophularaceous weeds typified by persian speedwell (Veronica persica); commelinaceous weeds typified by Asiatic dayflower (Commelina communis); labiatae weeds typified by henbit (Laminm amplexicaule) and purple deadnettle (Lamium purpureum); euphorbiaceous weeds typified by milk purslane (Euphorbia supina) and spotted spurge (Euphorbia maculata); rubiaceous weeds typified by bedstraw (Galium spurium), cleavers (Galium aparine) and madder (Rubia akane); violaceous weeds typified by violet (Viola arvensis); and leguminous weeds typified by hemp sesbania (Sesbania exaltata) and sicklepod (Cassia obtusifolia); graminaceous weeds typified by sorghum (Sorghum bicolor), fall panicum (Panicum dichotomiflorum), Johnsongrass (Sorghum halepense), barnyardgrass (Echinochloa crus-galli), henry crabgrass (Digitaria adscendens), wildoat (Avena fatua), goosegrass (Eleusine indica), green foxtail (Setaria viridis) and water foxtail (Alopecurus aequalis); and cyperaceous weeds typified by purple nutsedge (Cyperus rotundus, Cyperus esculentus).

Further, the compound of the present invention can be also used for any one of pre-emergence treatment and post-emergence treatment under submergence as a herbicide for paddy land. Examples of paddy weeds include alismataceous weeds typified by oriental waterplantain (Alisma canaliculatum), arrowhead (Sagittaria trifolla) and Sagittaria pygmaea, cyperaceous weeds typified by umbrella plant (Cyperus difformis), Cyperus serotinus, bulrush (Scirpus juncoides) and water chestnut (Eleochadaris kuroguwai); scrothulariaceous weeds typified by common falsepimpernel (Lindenia pyxidaria); potenderiaceous weeds typified by monochoria (Monochoria Vaginalis); potamogetonaceous weeds typified by largeleaf pondweed (Polgeton distinctus); lythraceous weeds typified by toothcup (Rotala indica); and graminaceous weeds typified by barnyardgrass (Echinochloa crus-galli).

The pyrazole derivative of the general formula (I), provided by the present invention, can be produced by the following method.

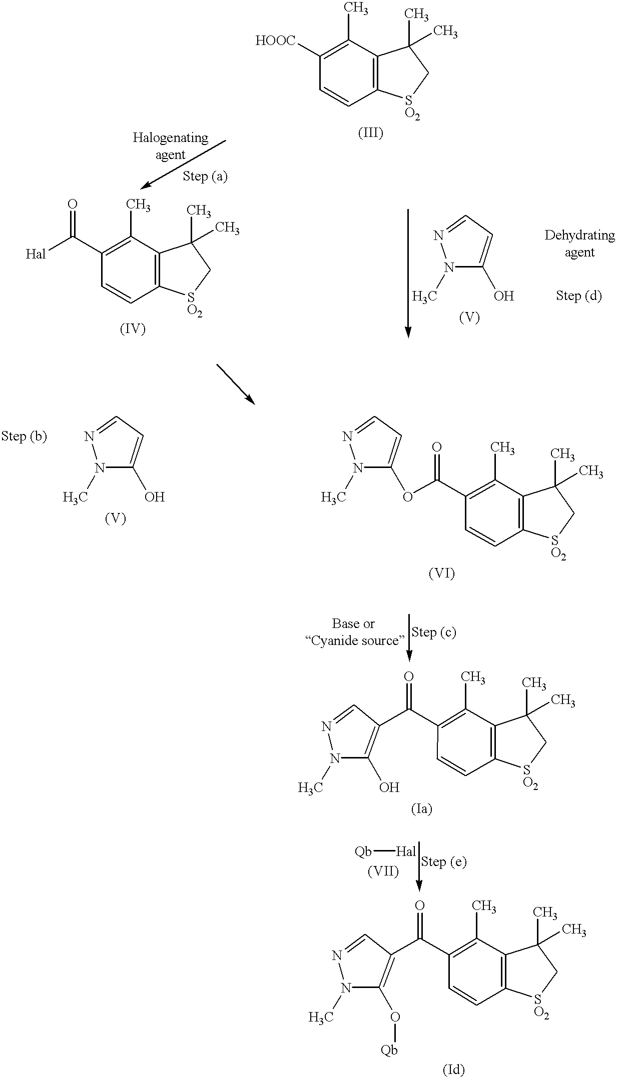

In the above reaction scheme, Qb is a group of —SO₂—R¹, —CO—R¹ or —CH₂CO—R¹, in which R¹ is a C₁-C₈ alkyl group, a C₃-C₈ cycloalkyl group, a C₁-C₈ haloalkyl group or a group of the formula (II),

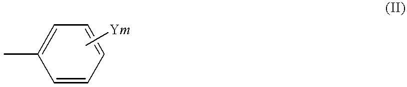

in which Y is a halogen atom, a nitro group, a C₁-C₄ alkyl group, a C₁-C₄ alkoxy group or a C₁-C₄ haloalkyl group, and m is an integer of 0 to 3, and Hal is a halogen atom.

That is, a compound of the formula (III) is reacted with a halogenating agent to obtain a compound of the general formula (IV), then, this compound is reacted with a compound of the formula (V) to obtain a compound of the formula (VI), and then this compound is subjected to a rearrangement reaction to obtain a pyrazole derivative of the formula (Ia). The pyrazole derivative of the formula (Ia) is reacted with a compound of the general formula Qb-Hal (VII), whereby a pyrazole derivative of the general formula (Id) can, be obtained. Further, the compound of the formula (VI) can be also obtained by a method in which the compound of the formula (III) is reacted with the compound of the formula (V) in the presence of a dehydrating agent such as dicyclohexylcarbodiimide (to be referred to as “DCC” hereinafter).

The above production method will be explained concerning each step hereinafter.

In the step (a), the compound of the formula (III) is reacted with a halogenating agent (thionyl chloride, phosphorus oxychloride, etc.) to obtain the compound of the formula (IV). In the step (a), it is preferred to use the halogenating agent in an equimolar amount or more based on the compound of the formula (III). The reaction may be carried out in a diluted state in an inert solvent (methylene chloride, chloroform, etc.), or it may be carried out without any solvent. Further, an excess of thionyl chloride as a halogenating agent may be used as a solvent. Although not specially limited, the reaction temperature is preferably 0° C. to the boiling point of the solvent, particularly preferably a temperature of 60° C. or around it.

In the step (b), the compound of the general formula (IV) obtained in the step (a) is reacted with the compound of the general formula (V) to obtain the compound of the formula (VI). In the step (b), preferably, the molar ratio of the compound of the general formula (IV)/compound of the general formula (V) is approximately 1/1 to 1/3, and the reaction is carried out in an inert solvent such as dioxane, acetonitrile, benzene, toluene, chloroform, methylene chloride or 1,2-dichloroethane. The reaction temperature is preferably 0° C. to 60° C., particularly preferably in the range of from 0° C. to room temperature.

Further, the compound (VI) can be also obtained by subjecting the compound (III) and the compound (V) to a dehydration in the presence of a dehydrating agent such as DCC (step (d)). The solvent used for the condensation is not specially limited so long as it is inert to the reaction, while it is preferably acetonitrile or tertiary amyl alcohol. The reaction temperature is not specially limited so long as it is in the range of from 0° C. to the boiling point of the solvent, while it is preferably room temperature. Besides the above DCC, the dehydrating agent can be selected from 1,1-carbonyldiimidazole (CDI) or 1-(3-dimethylaminopropyl)-3-ethylcarbodiimide (EDC). The amount of the dehydrating agent based on the compound (III) is preferably 1.0 to 3.0 equivalent amount, more preferably 1.0 to 1.5 equivalent amount. The compound (III)/compound (V) molar ratio is preferably in the range of from 1/1 to 1/3, more preferably 1/1 to 1/1.5. The reaction time for the condenstion of the above compounds is sufficiently in the range of 1 to 48 hours. Generally, the reaction is completed in approximately 8 hours.

In the step (c), the compound of the formula (VI) obtained in step (b) or (d) is subjected to a rearrangement reaction to obtain the pyrazole derivative of the general formula (Ia). In the step (c), preferably, the reaction is carried out in an inert solvent such as methylene chloride, 1,2-dichloroethane, toluene, acetonitrile, N,N-dimethylformamide or ethyl acetate. Acetonitrile is particularly preferred. In the step (c), a proper base (sodium carbonate, potassium carbonate, triethylamine or pyridine) is used generally in 1 to 4 equivalent amount, preferably in 1 to 2 equivalent amount, per the compound of the formula (VI). In this case, the reaction smoothly proceeds in the catalytic co-presence of hydrogen cyanide or a compound which can generate cyanide anion in the reaction system, a so-called “cyanide source”. The cyanide source is selected, for example, from metal cyanides such as sodium cyanide and potassium cyanide or cyanhydrin compounds of lower alkyl (C₃˜C₆) ketones such as acetonecyanhyrdin and methylisopropylketonecyanhydrin. When the metal cyanide is used, the reaction can be smoothly proceeded with by adding a phase transfer catalyst such as a crown ether during the reaction. The amount of the cyanide source per mole of the compound of the formula (V) is generally 0.01 to 0.5 mol, preferably 0.05 to 0.2 mol. The reaction temperature is preferably 0 to 80° C., particularly preferably 20 to 40° C.

In the step (e), the compound (Ia) obtained by the steps (a) to (d) is reacted with Qb-Hal(VII) (in which Qb is as defined above and Hal is a halogn atom) in the presence of a base in an inert solvent, to produce the pyrazole derivative (Id) of the general formula (I) in which Q is a group other than a hydrogen atom. In the above reaction, generally, it is preferred to use the compound (VII) in 1 to 3 equivalent amount per the pyrazole derivative (Ia). Further, for capturing hydrogen halide which the reaction produces as a by-product, it is preferred to use a base such as sodium carbonate, potassium carbonate, triethylamine or pyridine in an equimolar amount or more based on the starting raw material of the formula (Ia). The reaction temperature is preferably set in the range of from room temperature to the boiling point of the solvent. The solvent used for the reaction includes aromatic hydrocarbons such as benzene and toluene, ethers such as diethyl ether, ketones such as methyl ethyl ketone, and halogenated hydrocarbons such as dichloroethane, chloroform and dichloroethane. A two-phase solvent system containing any one of the above solvents and water may be used. In this case, a desirable result can be obtained by adding a phase transfer catalyst such as crown ether or benzyltriethylammonium chloride.

After the reaction, the reaction mixture is liquid-separated according to a conventional method, and the end product is obtained by extracting an aqueous phase with an organic solvent such as dichloromethane, dehydrating an organic layer and then distilling off the solvelnt, whereby the pyrazole derivative (Id) as an end product can be isolated.

Further, the starting material (III) used in the above scheme can be prepared by the following steps (1), (2), (3) and (4).

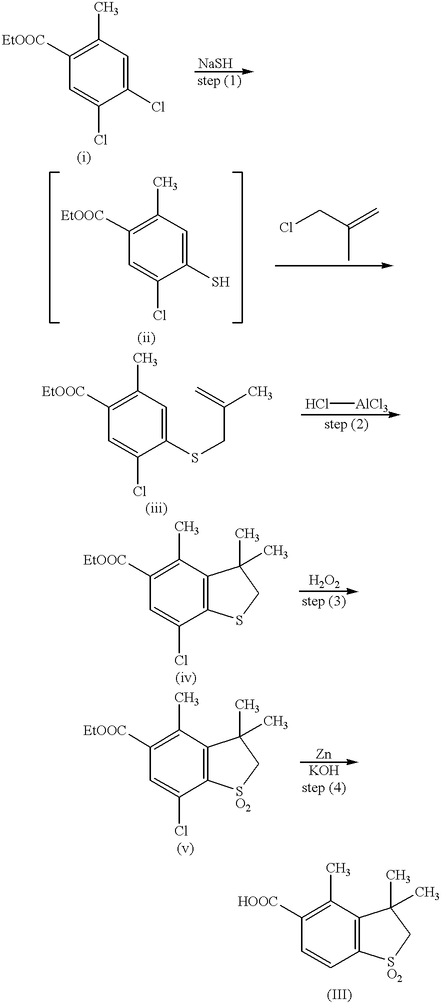

The reactions along the scheme will be explained in detail hereinafter. Ethyl 3,4-dichloro-6-methylbenzoate used as a starting material in the above scheme can be synthesized according to the method disclosed in WO97/08163.

Step (1)

First, benzoic acid ester of the formula (i) is reacted with sodium hydrosulfide, and then the reaction product is reacted with methallyl chloride to obtain a sulfide derivative of the formula (iii) via thiophenol of the formula (ii) as an intermediate. The condensation can be carried out in a solvent inert to the reaction, such as toluene, N-methylpyrrolidone or N,N-dimethylformamide, in the presence of a base such as sodium hydroxide, potassium hydroxide, potassium carbonate or sodium carbonate. The reaction temperature is from room temperature to the reflux temperature of the solvent, while it is preferably 80 to 130° C.

Step (2)

In the step (2), the sulfide derivative of the formula (iii) is condensed and cyclized to obtain a benzothiophene derivative of the formula (iv). As a cyclization method, there is a method in which the sulfide derivative is dehydratively cyclized in the presence of an acid catalyst such as aluminum chloride, hydrogen fluoride, sulfuric acid, phosphorus pentachloride, phosphoric acid, a polyphosphoric acid, tin chloride or zinc chloride. Any reaction solvent may be used without any limitation so long as it is inert under reaction conditions, while it can be selected from hydrocarbon solvents such as pentane and hexane or halogen-containing solvents such as dichloromethane and 1,2-dichloroethane.

Step (3)

In the step (3), the benzothiophene derivative of the formula (iv) is oxidized to obtain a benzothiophene 1,1-dioxide derivative of the formula (v). As an oxidation method, there is a method using hydrogen peroxide or an organic peroxide such as m-chloroperbenzoic acid. Any reaction solvent may be used without any limitation so long as it is inert under reaction conditions, while it can be selected from lower carboxylic acid such as acetic acid, hydrocarbon solvents such as pentane and hexane or halogen-containing solvents such as dichloromethane or 1,2-dichloroethane.

Step (4)

In the step (4), the benzothiophene 1,1,-dioxide derivative of the formula (v) is reductively dechlorinated to obtain a carboxylic acid derivative of the formula (vi). The reducing method is not specially limited. For example, the reduction is achieved by a method using a metal reducing agent such as powdered zinc in a solvent inert to the reaction such as an alcohol, or by a method in which hydrogenation is carried out in the presence of a reducing catalyst such as palladium or nickel under atmospheric pressure or elevated pressure. Preferably, ethanol-water are used as a solvent, and the reaction is carried out in the presence of powdered zinc at a reaction temperature of 0° C. to room temperature.

Table 1 shows specific examples of the pyrazole derivative of the present invention obtained as described above. In Table 1, “Pr” stands for propyl group, “Bu” stands for butyl group, and “c” stands for cyclic.

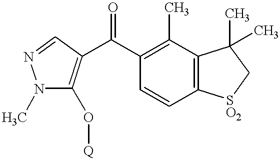

| Compound | | Compound | |
|---|---|---|---|
| No. | Q | No. | Q |
| 1 | H | 28 | CH₃CO |
| 2 | CH₃SO₂ | 29 | C₂H₅CO |
| 3 | C₂H₅SO₂ | 30 | n-PrCO |
| 4 | n-PrSO₂ | 31 | i-PrCO |
| 5 | i-PrSO₂ | 32 | c-PrCO |
| 6 | c-PrSO₂ | 33 | n-BuCO |
| 7 | n-BuSO₂ | 34 | i-BuCO |
| 8 | i-BuSO₂ | 35 | s-BuCO |
| 9 | s-BuSO₂ | 36 | t-BuCO |
| 10 | t-BuSO₂ | 37 | c-C₆H₁₁CO |
| 11 | c-C₆H₁₁SO₂ | 38 | n-C₈H₁₇CO |
| 12 | n-C₈H₁₇SO₂ | 39 | C₆H₅CO |
| 13 | ClCH₂SO₂ | 40 | o-CH₃C₆H₄CO |
| 14 | ClCH₂CH₂CH₂SO₂ | 41 | m-CH₃C₆H₄CO |
| 15 | C₆H₅SO₂ | 42 | p-CH₃C₆H₄CO |
| 16 | o-CH₃C₆H₄SO₂ | 43 | p-FC₆H₄CO |
| 17 | m-CH₃C₆H₄SO₂ | 44 | p-ClC₆H₄CO |
| 18 | p-CH₃C₆H₄SO₂ | 45 | p-Br₆H₄CO |
| 19 | p-C₆H₄SO₂ | 46 | p-IC₆H₄CO |
| 20 | p-FC₆H₄SO₂ | 47 | p-CH₃OC₄H₄CO |
| 21 | p-ClC₆H₄SO₂ | 48 | p-NO₂C₆H₄CO |
| 22 | p-BrC₆H₄SO₂ | 49 | C₆H₅COCH₂ |
| 23 | p-IC₆H₄SO₂ | 50 | (C₂H₅)₃NH⁺ |
| 24 | 2,4-Cl2C₆H₃SO₂ | 51 | iPrNH₅⁺ |
| 25 | p-CH₃OC₆H₄SO₂ | 52 | Na |
| 26 | p-NO₂C₆H₄SO₂ | | |
| 27 | 2,4,6-(CH₃)₃C₆H₂SO₂ | | |

The pyrazole derivative of the present invention is free of phytotoxicity to corn and can control a broad range of cropland weeds at a low dosage.

The present invention will be explained in detail with reference to Preparation Examples and Herbicide Examples hereinafter, while the present invention shall not at all be limited by these Examples.

### REFERENTIAL PREPARATION EXAMPLE STEP (1))

### Synthesis of (2-chloro-4-ethoxycarbonyl-5-methylphenyl)(2-methyl-2-propene)sulfide

10 Grams (43 mmol) of ethyl 3,4-dichloro-6-methylbenzoate and 8.53 g (2.5 equivalent amount, 0.11 mol) of 70 wt % sodium hydrosulfide were suspended in 40 ml of DMF (dimethylformamide), and the mixture was stirred under a nitrogen gas current at 80° C. for 3 hours. The reaction mixture was cooled, and ice water, ethyl acetate and 15 ml of concentrated hydrochloric acid were consecutively added. Then, the mixture was separated into two phases. An organic layer was washed with 5% hydrochloric acid three times, then washed with a saturated sodium chloride aqueous solution, and then dried over anhydrous sodium sulfate. The solvent was distilled off under reduced pressure, then, the remainder was dissolved in 40 ml of acetone, 5.93 g (1.0 equivalent amount, 43 mmol) of potassium carbonate was added, and the mixture was cooled in an ice water bath. To the mixture was added 4.7 ml (1.1 equivalent amount, 48 mmol) of methallyl chloride, and the mixture was stirred for 10 minutes as it was, and then stirred for 30 minutes while bringing its temperature back to room temperature. Then, the mixture was refluxed under heat for 1 hour. The acetone was distilled off under reduced pressure, water was added, and a reaction product was extracted with ethyl acetate and washed with a saturated sodium chloride aqueous solution and dried over anhydrous sodium sulfate. The solvent was distilled off under reduced pressure to give 11.0 g (yield 80%) of crude (2-chloro-4-ethoxycarbonyl-5-methylphenyl)(2-methyl-2-propene)sulfide.

¹H-NMR (chloroform-d): 1.37 (t, 3H), 1.87 (s, 3H), 2.57 (s, 3H), 3.59 (s, 2H), 4.30 (q, 2H), 4.93 (s, 1H), 5.00 (s, 1H), 6.98 (s, 1H), 7.22 (s, 1H), 7.87 (s, 1H)

### REFERENTIAL PREPARATION EXAMPLE 2 (STEP (2))

### Synthesis of 7-chloro-5-ethoxycarbonyl-3,3,4-trimethyl-2,3-dihydrobenzothiophene

8.44 Grams (3.0 equivalent amount, 63 mmol) of aluminum chloride was suspended in 70 ml of methylene chloride, and while hydrochloric acid gas was introduced into the reaction system, the suspension was stirred at room temperature for 10 minutes and stirred for 10 minutes while cooling it to 0° C. To the reaction mixture was dropwise added a solution of 6.0 g (−21 mmol) of (2-chloro-4-ethoxycarbonyl-5-methylphenyl)(2-methyl-2-propene)sulfide in 20 ml of methylene chloride, and the mixture was stirred for 10 minutes as it was. Then, the mixture was stirred for 3 hours while bringing its temperature back to room temperature. The reaction mixture was added to ice, and a reaction product was extracted with methylene chloride twice and dried over anhydrous sodium sulfate. The solvent was distilled off under reduced pressure to give 5.50 g of crude 7-chloro-5-ethoxycarbonyl-3,3,4-trimethyl-2,3-dihydrobenzothiophene. The crude product was purified by silica gel column chromatography, to give 3.67 g (yield 60%) of the intended product.

¹H-NMR (chloroform-d): 1.30 (t, 3H), 1.52 (s, 6H), 2.52 (s, 3H), 3.17 (s, 2H), 4.30 (q, 2H), 7.58 (s, 1H)

### REFERENTIAL PREPARATION EXAMPLE 3 (STEP (3))

### Synthesis of 7-chloro-5-ethoxycarbonyl-3,3,4-trimethyl-2,3-dihydrobenzothiophene-1,1-dioxide

6.7 Grams (24 mmol) of 7-chloro-5-ethoxycarbonyl-3,3,4-trimethyl-2,3-dihydrobenzothiophene was dissolved in 13 ml of 1,2-dichloroethane, and 4.8 ml (3.5 equivalent amount, 84 mmol) of acetic acid and 5.6 ml (2.3 equivalent amount, 55 mmol) of a 30 wt % hydrogen peroxide aqueous solution were consecutively added. The mixture was stirred at 80° C. for 3.5 hours. Ice water was added to the reaction mixture, and then a solution of 3.0 g (23 mmol) of sodium sulfite in 30 ml of water was added. A reaction product was extracted with ethyl acetate, washed with a sodium carbonate aqueous solution twice, washed with a saturated sodium chloride aqueous solution and dried over anhydrous sodium sulfate. The solvent was distilled off under reduced pressure to give 7.21 g of crude 7-chloro-5-ethoxycarbonyl-3,3,4-trimethyl-2,3-dihydrobenzothiophene-1,1-dioxide. The crude product was purified by silica gel column chromatography to give 6.40 g (yield 90%) of the intended product.

¹H-NMR (chloroform-d): 1.43 (t, 3H), 1.65 (s, 6H), 2.57 (s, 3H), 3.43 (s, 2H), 4.39 (q,2H), 7.67 (s, 1H)

### REFERENTIAL PREPARATION EXAMPLE 4 (STEP (4))

### Synthesis of 5-carboxyl-3,3,4-trimethyl-2,3-dihydrobenzothiophene-1,1-dioxide

6.56 Grams (21 mmol) of 7-chloro-5-ethoxycarbonyl-3,3,4-trimethyl-2,3-dihydrobenzothiophene-1,1-dioxide was dissolved in 26 ml of ethanol, and 20 ml of a 20 wt % potassium hydroxide aqueous solution and 3.38 g (2.5 equivalent amount, 52 mmol) of a zinc powder were consecutively added. The mixture was stirred at 80° C. for 4 hours. Water was added, and the zinc was filtered off. Then, a reaction product was extracted from a filtrate with ethyl acetate, and consecutively washed with 5% hydrochloric acid and with a saturated sodium chloride aqueous solution, and dried over anhydrous sodium sulfate. The solvent was distilled off to give 5.75 g (yield, quantitative) of crude 5-carboxyl-3,3,4-trimethyl-2,3-dihydrobenzothiophene-1,1-dioxide.

¹H-NMR (chloroform-d): 1.70 (s, 3H), 2.72 (s, 3H), 3.37 (s, 2H), 7.64 (d, 1H), 7.98 (d, 1H)

Preparation Examples will be described below.

### PREPARATION EXAMPLE 1

### 5-(1′-methyl-5′-hydroxypyrazol-4′-yl)carbonyl-3,3,4-trimethyl-2,3-dihydrobenzothiophene-1,1-dioxide (Compound 1)

1.0 Gram (3.9 mmol) of 5-carboxyl-3,3,4-trimethyl-2,3-dihydrobenzothiophene-1,1-dioxide was dissolved in 10 ml of dichloroethane, and 0.95 g (2.0 equivalent amount, 8.0 mmol) of thionyl chloride was added. The mixture was stirred under heat at 60° C. for 30 minutes. Excess thionyl chloride and dichloroethane were distilled off, and the resultant acid chloride was dissolved in 10 ml of acetonitrile. Then, 0.96 g (2.4 equivalent amount, 9.5 mmol) of triethylamine and 0.56 g (1.1 equivalent amount, 4.2 mmol) of 1-methyl-5-hydroxypyrazole hydrochloride were added, and the mixture was stirred at room temperature for 4 hours. Then, 0.48 g (1.2 equivalent amount, 4.7 mmol) of triethylamine and 3 drops of acetone cyanhydrin were added, and the mixture was stirred at room temperature for 1 day. After completion of the reaction, a reaction product was extracted with a 2% potassium carbonate aqueous solution, and an aqueous layer was washed with methylene chloride. The aqueous layer was neutralized with 5% hydrochloric acid, and subjected to extraction with ethyl acetate. An organic layer was washed with a saturated sodium chloride aqueous solution and dried over anhydrous sodium sulfate. The solvent was distilled off, to give 0.88 g (yield 67%) of the subject end product.

### PREPARATION EXAMPLE 2

### 5-(1′-methyl-5′-n-propanesulfonyloxypyrazol-4′-yl)carbonyl-3,3,4-trimethyl-2,3-dihydrobenzothiophene-1,1-dioxide (Compound 4)

0.48 Gram (1.4 mmol) of 5-(1′-methyl-5′-hydroxypyrazol-4′-yl)carbonyl-3,3,4-trimethyl-2,3-dihydrobenzothiophene-1,1-dioxide was dissolved in 5 ml of methylene chloride, and 5 ml of water, 0.40 g (2.1 equivalent amount, 2.9 mmol) of potassium carbonate, 0.40 g (2.0 equivalent amount, 2.8 mmol) of n-propanesulfonyl chloride and benzyltriethylammonium chloride (catalytic amount) were added. The mixture was stirred at room temperature for 1 day. After completion of the reaction, a methylene chloride layer was recovered and dried over anhydrous sodium sulfate, and the solvent was distilled off under reduced pressure. The resultant oil was purified by silica gel column chromatography to give 0.43 g (yield 68%) of 5-(1′-methyl-5′-n-propanesulfonyloxypyrazol-4′-yl)carbonyl-3,3,4-trimethyl-2,3-dihydrobenzothiophene-1,1-dioxide.

Sulfonic acid esters as Compounds 2, 3, 5, 7, 8, 12 to 15, 18, 19, 21 and 24 to 27 were obtained in the same manner as in the above Preparation Example except that the n-propanesulfonyl chloride was replaced with corresponding sulfonyl chlorides.

### PREPARATION EXAMPLE 3

### 5-(1′-methyl-5′-n-propionyloxypyrazol-4′-yl)carbonyl-3,3,4-trimethyl-2,3-dihydrobenzothiophene-1,1-dioxide (Compound 30)

0.50 Gram (1.5 mmol) of 5-(1′-methyl-5′-hydroxypyrazol-4′-yl)carbonyl-3,3,4-trimethyl-2,3-dihydrobenzothiophene-1,1-dioxide was dissolved in 5 ml of methylene chloride, and 0.18 g (1.2 equivalent amount, 1.8 mmol) of triethylamine and 0.17 g (1.2 equivalent amount, 1.8 mmol) of propionyl chloride were added. The mixture was stirred at room temperature for 1 day. After completion of the reaction, the solvent was distilled off under reduced pressure. The resultant oil was purified by silica gel column chromatography to give 0.28 g (yield 48%) of 5-(1′-methyl-5′-n-propionyloxypyrazol-4′-yl)carbonyl-3,3,4-trimethyl-2,3-dihydrobenzothiophene-1,1-dioxide.

Carboxylic acid esters as Compounds 29, 32, 37, 39 and 42 were obtained in the same manner as in the above Preparation Example except that the propionyl chloride was replaced with corresponding carboxylic acid chlorides.

### PREPARATION EXAMPLE 4

### 5-(1′-methyl-5′-phenacyloxypyrazol-4′-yl)carbonyl-3,3,4-trimethyl-2.3-dihydrobenzothiophene-1,1-dioxide (Compound 49)

0.50 Gram (1.5 mmol) of 5-(1′-methyl-5′-hydroxypyrazol-4′-yl)carbonyl-3,3,4-trimethyl-2,3-dihydrobenzothiophene-1,1-dioxide was dissolved in 5 ml of acetone, and 0.31 g (1.5 equivalent amount, 2.2 mmol) of potassium carbonate and 0.45 g (1.5 equivalent amount, 2.3 mmol) of phenacyl bromide were added. The mixture was stirred at room temperature for 1 day. After completion of the reaction, a methylene chloride layer was recovered and dried over anhydrous sodium sulfate. The solvent was distilled off under reduced pressure, and the resultant oil was purified by silica gel column chromatography to give 0.56 g (yield 83%) of 5-(1′-methyl-5′-n-phenacyloxypyrazol-4′-yl)carbonyl-3,3,4-trimethyl-2,3-dihydrobenzothiophene-1,1-dioxide.

### PREPARATION EXAMPLE 5

### Triethylamine Salt (Compound 50)

0.50 Gram (1.5 mol) of 5-(1′-methyl-5′-hydroxypyrazol-4′-yl)carbonyl-3,3,4-trimethyl-2,3-dihydrobenzothiophene-1,1-dioxide was dissolved in 10 ml of acetonitrile, and 0.29 g (2.9 mmol) of triethylamine was added. The solvent was distilled off under reduced pressure to give 0.64 g of 5-(1′-methyl-5′-hydroxypyrazol-4′-yl)carbonyl-3,3,4-trimethyl-2,3-dihydrobenzothiophene-1,1-dioxide triethylamine salt.

Isopropylamine salt as Compound 51 was obtained in the same manner as in Preparation Example 5 except that the triethylamine was replaced with isopropylamine.

### PREPARATION EXAMPLE 6

### Sodium Salt (Compound 52)

1.0 Gram (3.0 mmol) of 5-(1′-methyl-5′-hydroxypyrazol-4′-yl)carbonyl-3,3,4-trimethyl-2,3-dihydrobenzothiophene-1,1-dioxide was dissolved in 10 ml of tetrahydrofuran, and 0.12 g (3.0 mmol) of sodium hydride (60 wt %, oily, washed with n-hexane) was added. A formed precipitate was recovered by filtration and washed with ethyl acetate to give 0.27 g (yield 25%) of 5-(1′-methyl-5′-hydroxypyrazol-4′-yl)carbonyl-3,3,4-trimethyl-2,3-dihdyrobenzothiophene-1,1-dioxide sodium salt.

Tables 2 to 6 show physical property values of compounds obtained in Preparation Examples.

TABLE 2

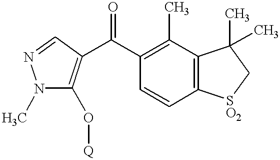

| Compound | | Yield | ¹H-NMR | IR | |
|---|---|---|---|---|---|
| No. | Q | (%) | (chloroform-d) | (KBr, cm⁻¹) | mp ° C. |
| 1 | H | 67 | 1.70(s, 6H), 2.50(s, 3H), | 3000, 1660, | 260.0- |
| | | | 3.38(s, 2H), 3.73(s, 3H), | 1540, 1300, | 267.7 |
| | | | 7.32(s, 1H), 7.53(d, 1H), | 1130. | |
| | | | 1.64(d, 1H). | | |
| 2 | CH₃SO₂ | 66 | 1.69(s, 6H), 2.45(s, 3H), | 2990, 2950, | |
| | | | 3.38(s, 2H), 3.57(s, 3H), | 1670, 1550, | |
| | | | 3.90(s, 3H), 7.44(s, 1H), | 1300, 1180. | |
| | | | 7.46(d, 1H), 7.62(d, 1H) | 1130. | |
| 3 | C₂H₅SO₂ | 76 | 1.63(t, 3H), 1.68(s, 6H), | 3000, 2980, | 156.6- |
| | | | 2.45(s, 3H), 3.37(s, 2H), | 1610, 1550, | 158.7 |
| | | | 3.67(q, 2H), 3.57(s, 3H), | 1300, 1180, | |
| | | | 3.90(s, 3H), 7.45(d, 1H), | 1130. | |
| | | | 7.46(s, 1H), 7.62(d, 1H). | | |
| 4 | n-PrSO₂ | 68 | 1.18(t, 3H), 1.69(s, 6H), 1.9- | 2970, 2930, | 150.8- |
| | | | 2.4(m, 2H), 2.46(s, 3H), | 1660, 1550, | 153.8 |
| | | | 3.38(s, 2H), 3.5-3.8(m, 2H), | 1370, 1300, | |
| | | | 3.90(s, 3H), 4.23(q, 2H), | 1170, 1120. | |
| | | | 7.38(d, 1H), 7.46(s, 1H), | | |
| | | | 7.61(d, 1H). | | |
| 5 | i-PrSO₂ | 60 | 1.59(d, 6H), 1.68(s, 6H), | 3480, 3450, | 152.3 |
| | | | 2.46(s, 3H), 3.37(s, 2H), | 3020, 2970 | 159.3 |
| | | | 3.84(sep, 1H), 3.90(s, 3H), | 1670, 1555, | |
| | | | 7.43(d, 1H), 7.51(s, 1H), | 1390, 1375, | |
| | | | 7.64(8, 1H). | 1310, 1185, | |
| | | | 1130. | | |

TABLE 3

| Compound | | Yield | | | |
|---|---|---|---|---|---|
| No. | Q | (%) | ¹H-NMR (chloroform-d) | IR (KBr, cm⁻¹) | mp ° C. |
| 7 | n-BuSO₂ | 93 | 1.01(t, 3H), 1.69(s, 6H), 1.5- | 2990, 1670, | |
| | | | 2.2(m, 4H), 2.45(s, 3H), | 1550, 1310, | |
| | | | 3.38(s, 2H), 3.6-3.8(m, 2H), | 1130, 1180. | |
| | | | 3.90(s, 3H), 7.45(s, 1H), | | |
| | | | 7.46(d, 1H), 7.61(d, 1H). | | |
| 8 | i-BuSO₂ | 82 | 1.21(d, 6H), 1.69(s, 6H), | 2980, 1670, | |
| | | | 2.45(s, 3H), 2.3-2.6(m, 1H), | 1540, 1300, | |
| | | | 3.38(s, 2H), 3.63(d, 1H), | 1120, 1160, | |
| | | | 3.90(s, 3H), 7.45(s, 1H), | | |
| | | | 7.46(d, 1H), 7.62(d, 1H). | | |
| 12 | n-C₈H₁₇SO₂ | 60 | 0.88(t, 3H), 1.20-1.52(m, 12H), | 3500, 2940, | |
| | | | 1.65(s, 3H), 2.44(s, 3H), | 1660, 1540, | |
| | | | 3.37(s, 2H), 3.60-3.77(m, 2H), | 1380, 1300, | |
| | | | 3.90(s, 3H), 7.41(d, 1H), | 1170, 1130. | |
| | | | 7.43(s, 1H), 7.65(d, 1H). | | |
| 13 | ClCH₂SO₂ | 42 | 1.69(s, 6H), 2.45(s, 3H), | 2950, 1660, | 186.2- |
| | | | 3.38(s, 2H), 3.92 (s, 3H), | 1545, 1410, | 187.0 |
| | | | 5.36(s, 2H), 7.43(d, 1H), | 1400, 1300, | |
| | | | 7.43(s, 1H), 7.67(d, 1H). | 1180, 1120. | |
| 14 | ClCH₂CH₂CH₂SO₂ | 55 | 1.68(s, 6H), 2.44(s, 3H), 2.3- | 2950, 1660, | |
| | | | 2.7(m, 2H), 3.38(s, 2H), 3.7- | 1545, 1380, | |
| | | | 4.0(m, 4H), 3.90(s, 3H), | 1300, 1170, | |
| | | | 7.41(d, 1H), 7.44(s, 1H), | 1120. | |
| | | | 7.66(d, 1H). | | |
| 15 | C₆H₅SO₂ | 79 | 1.68(s, 6H), 2.43(s, 3H), | 2950, 1660, | |
| | | | 3.37(s, 2H), 3.81(s, 3H), 7.5- | 1545, 1395, | |
| | | | 8.07(m, 8H). | 1370, 1300, | |
| | | | | 1205, 1180, | |
| | | | | 1125. | |

TABLE 4

| Compound | | Yield | | | |
|---|---|---|---|---|---|
| No. | Q | (%) | ¹H-NMR (chloroform-d) | IR (KBr, cm⁻¹) | mp ° C. |
| 18 | p-CH₃C₆H₄SO₂ | 68 | 1.68(s, 6H), 2.44(s, 3H), | 3000, 1670, | |
| | | | 2.45(s, 3H), 3.37(s, 2H), 3.79 | 1550, 1300, | |
| | | | (s, 3H), 7.23-7.77(m, 7H). | 1130. | |
| 19 | p-C₂H₅C₆H₄SO₂ | 53 | 2.24(t, 3H) 1.68(s, 6H), | 2980, 1650, | 137.1- |
| | | | 2.44(s, 3H), 2.75(q, 2H), | 1540, 1365, | 139.2 |
| | | | 3.37(s, 2H), 3.80(s, 3H), 7.2- | 1290, 1200, | |
| | | | 7.8(m, 7H). | 1170, 1120. | |
| 21 | p-ClC₆H₄SO₂ | 85 | 1.68(s, 3H), 2.42(s, 3H), | 3000, 2950, | 191.6- |
| | | | 3.38(s, 2H), 3.86(s, 3H), | 1650, 1550, | 193.6 |
| | | | 7.31(d, 1H), 7.56(s, 1H), 7.6-7.7 | 1400, 1380, | |
| | | | (m, 3H), 7.8-7.9(m, 2H). | 1305, 1210, | |
| | | | | 1185, 1130, | |
| | | | | 1090. | |
| 24 | 2,4-Cl₂C₆H₃SO₂ | 79 | 1.66(s, 6H), 2.34(s, 3H), | 3000, 1680, | 207.9- |
| | | | 3.35(s, 2H), 3.91(s, 3H), 7.2-7.9 | 1560, 1410, | 210.0 |
| | | | (m, 6H). | 1310, 1190, | |
| | | | | 1140. | |
| 25 | p-CH₃OC₆H₄SO₂ | 61 | 1.69(s, 6H), 2.46(s, 3H), | 2960, 1660, | 182.4- |
| | | | 3.38(s, 2H), 3.81(s, 3H), 3.90 | 1600, 1530, | 183.4 |
| | | | (s, 3H), 7.0-7.8(m, 7H). | 1370, 1300, | |
| | | | | 1170, 1120. | |
| 26 | p-NO₂C₆H₄SO₂ | 90 | 1.67(s, 6H), 2.38(s, 3H), | 3100, 2980, | 216.9- |
| | | | 3.37(s, 2H), 3.94(s, 3H), | 1665, 1535, | 217.4 |
| | | | 7.31(d, 1H), 7.50(s, 1H), | 1410, 1290, | |
| | | | 7.61(d, 1H), 8.24(d, 2H), | 1190, 1120. | |
| | | | 8.49(d, 2H). | | |
| 27 | 2,4,6- | 77 | 1.65(s, 6H), 2.31(s, 3H), | 3130, 2980, | 184.4- |
| | (CH₃)₃C₆H₂SO₂ | | 2.33(s, 3H), 2.59(s, 6H), | 1670, 1540, | 188.0 |
| | | | 3.34(s, 2H), 3.77(s, 3H), | 1380, 1300, | |
| | | | 7.02(s, 2H), 7.19(d, 1H), | 1180, 1130. | |
| | | | 7.43(s, 1H), 7.56(d, 1H). | | |

TABLE 5

| Compound | | Yield | | | |
|---|---|---|---|---|---|
| No. | Q | (%) | ¹H-NMR (chloroform-d) | IR (KBr, cm⁻¹) | mp ° C. |
| 29 | C₂H₅CO | 48 | 2.22(t, 3H), 1.68(s, 6H), | 3000, 1800, | |
| | | | 2.41(s, 3H), 2.53(q, 2H), | 1660, 1550, | |
| | | | 3.37(s, 2H), 3.72(s, 3H), | 1300, 1120. | |
| | | | 7.43(d, 1H), 7.60(s, 1H), | | |
| | | | 7.60(d, 1H). | | |
| 30 | n-PrCO | 42 | 1.11(t, 3H), 1.6-1.9(m, 2H), | 2980, 1790, | |
| | | | 1.60(s, 6H), 2.41(s, 3H), | 1660, 1530, | |
| | | | 2.47(t, 2H), 3.37(s, 2H), | 1370, 1300, | |
| | | | 3.72(s, 3H), 7.43(d, 1H), | 1120, 1060. | |
| | | | 7.60(s, 1H), 7.60(d, 1H). | | |
| 32 | c-C₃H₅CO | 82 | 1.20(d, 4H), 1.60-1.74(m, 1H), | 2950, 1780, | |
| | | | 1.68(s, 6H), 2.41(s, 3H), | 1650, 1550, | |
| | | | 3.36(s, 2H), 3.70(s, 3H), | 1300, 1140, | |
| | | | 7.36(d, 1H), 7.63(d, 1H), | 1120. | |
| | | | 7.77(s, 1H). | | |
| 37 | c-C₄H₁₁CO | 62 | 1.1-2.0(m, 11H), 1.65(s, 6H), | 2950, 1780, | 146.2- |
| | | | 2.41(s, 3H), 3.34(s, 2H), | 1705, 1650, | 171.0 |
| | | | 3.68(s, 3H), 7.40(d, 1H), | 1550, 1450, | |
| | | | 7.59(s, 1H), 7.60(d, 1H). | 1300, 1120. | |
| 39 | C₆H₅CO | 29 | 1.45(s, 6H), 2.36(s, 3H), | 2990, 2950, | |
| | | | 3.10(s, 2H), 3.35(s, 3H), 7.2- | 1770, 1650, | |
| | | | 7.9(m, 8H). | 1300, 1120. | |
| 42 | p-CH₃C₆H₄CO | 51 | 1.46(s, 6H), 2.35(s, 3H), 2.46(s, | 2950, 1760, | 184.8- |
| | | | 3H), 3.08(s, 2H), 3.74(s, 3H), | 1640, 1300, | 194.4 |
| | | | 7.2-7.9(m, 7H). | 1245, 1180, | |
| | | | | 1120. | |
| 49 | C₆H₅ CO CH₂ | 83 | 1.68(s, 6H), 2.41(s, 3H), | 3140, 3080, | |
| | | | 3.37(s, 2H), 3.56(s, 3H), | 3000, 1800, | |
| | | | 3.82(s, 2H), 7.32(s, 1H), | 1660, 1540, | |
| | | | 7.35(s, 5H), 7.40(d, 1H), | 1300, 1180. | |
| | | | 7.66(d, 1H). | | |
| 50 | Et₃NH⁺ | 99 | | 3000, 2950, | |
| | | | | 1630, 1510, | |
| | | | | 1300, 1130. | |

TABLE 6

| Compound | | Yield | | | |
|---|---|---|---|---|---|
| No. | Q | (%) | ¹H-NMR (chloroform-d) | IR (KBr, cm⁻¹) | mp ° C. |
| 51 | i-PrNH₃* | 99 | 1.09(d, 6H), 1.63(s, 6H), | 3000, 1620, | 102.1- |
| | | | 2.40(s, 3H), 3.14(sep, 1H), | 1510, 1390, | 106.1 |
| | | | 3.36(s, 2H), 3.44(s, 3H), | 1310, 1130, | |
| | | | 6.92(s, 1H), 7.24(d, 1H), | 930. | |
| | | | 7.56(d, 1H). | | |
| 52 | Na | 25 | | 3500, 1630, | |
| | | | | 1450, 1300, | |
| | | | | 1120. | |

Herbicide Examples will be described below.

(1) Preparation of Herbicides

97 Parts by weight of talc (trade name: Zeaklite, supplied by Zeaklite Industry) as a carrier, 1.5 parts by weight of alkylarylsulfonic acid salt(trade name: Neoplex, supplied by Kao-Atlas K.K.) as a surfactant and 1.5 parts by weight of a nonionic and anionic surfactant (trade name: Sorpol 800A, supplied by Toho Chemical Co., Ltd.) were uniformly pulverized and mixed to prepare a carrier for a wettable powder.

90 Parts by weight of the above carrier for a wettable powder and 10 parts by weight of one of the compounds of the present invention were uniformly pulverized and mixed to obtain herbicides. Further, in Comparative Herbicide Examples, comparative herbicides were also prepared from the following compounds (A) and (B) in the same manner.

Compound (A): Compound disclosed in WO96/25412

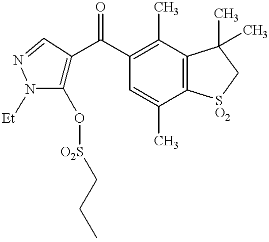

Compound (B): Compound disclosed in WO97/08164

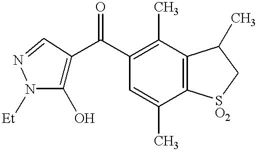

(2) Ratings of Evaluation of Herbicidal Efficacy and Phytotoxicity To Crops

Herbicidal efficacy and phytotoxicity to crops were determined on the basis of the ratio of remaining plant weight to plant weight in non-treated plot=(remaining plant weight in treated plot/plant weight in non-treated-plot)×100. The ratings were applied to the following biological tests.

Ratings

| | Ratio of remaining plant |
|---|---|
| | weight to plant weight |
| | in non-treated plot (%) |
| Herbicidal efficacy | |
| 0 | 81-100 |
| 1 | 61-80 |
| 2 | 41-60 |
| 3 | 21-40 |
| 4 | 1-20 |
| 5 | 0 |
| Phytotoxicity | |
| to crops | |
| − | 100 |
| ± | 95-99 |
| + | 90-94 |
| ++ | 80-89 |
| +++ | 0-79 |

(3) Biological Tests (Upland Pre-emergence Treatment Test, Compounds Nos. 1-4, 18, 30, 37, 39, 49, 50 and 52, and Compounds (A) and (B))

Seeds of weeds such as velvetleaf, common lambsquaters, redroot pigweed, common ragweed, green foxtail and large crabgrass and seeds of corn were sown in 1/5,000-are Wagner pots filled with upland soil, and covered with upland soil. Then, a predetermined amount of the herbicide prepared in the above (1) was suspended in water, and the suspension was uniformly sprayed onto the soil surface at a rate of 2,000 liters/hectare. Then, the seeds were grown in a greenhouse, and on the 20th day after the treatment, the herbicide was evaluated for herbicidal efficacy and phytotoxicity to corn on the basis of the ratings shown in (2). Table 7 shows the results.

TABLE 7

| Com- | | | | | | | | Phyto- |
|---|---|---|---|---|---|---|---|---|
| pound | Dosage | Herbicidal Efficacy | | | | | | toxicity |
| No. | g/ha | AA | BB | CC | DD | EE | FF | Corn |
| 1 | 80 | 5 | 5 | 5 | 5 | 5 | 5 | − |
| 2 | 80 | 5 | 5 | 5 | 5 | 5 | 5 | − |
| 3 | 80 | 5 | 5 | 5 | 5 | 5 | 5 | − |
| 4 | 80 | 5 | 5 | 5 | 5 | 5 | 5 | − |
| 18 | 80 | 5 | 5 | 5 | 4 | 5 | 5 | − |
| 30 | 80 | 4 | 5 | 5 | 5 | 5 | 5 | − |
| 37 | 80 | 5 | 5 | 5 | 5 | 5 | 5 | − |
| 39 | 80 | 5 | 5 | 5 | 5 | 5 | 5 | − |
| 49 | 80 | 5 | 5 | 5 | 5 | 5 | 5 | − |
| 50 | 80 | 5 | 5 | 5 | 4 | 5 | 5 | − |
| 52 | 80 | 4 | 5 | 5 | 4 | 4 | 5 | − |
| (A) | 80 | 2 | 2 | 0 | 1 | 1 | 1 | − |
| (B) | 80 | 1 | 4 | 1 | 2 | 5 | 5 | − |

Notes: AA = Velvetleaf, BB = Common lambsquaters, CC = Rough Pigweed, DD = Common ragweed, EE = Green foxtail, FF = Large crabgrass

The results in Table 7 show that the herbicides of the present invention cause no phytotoxicity on corn and can selectively control a broad range of principal cropland weeds from monocotyledonous weeds to dicotyledonous weeds at a low dosage. On the other hand, it is shown that Compound (A) is clearly inferior in herbicidal efficacy, particularly clearly poor in redroot pigweed. It is also shown that Compound (B) is inferior in herbicidal efficacy against velvet leaf, common ragweed and redroot pigweed.

(4) Biological Tests (Upland Post-mergence Treatment Test. Compounds Nos. 1-8, 12-15, 18, 19, 21, 24-27, 29, 30, 37, 39, 42 and 49-52, and Compounds (A) and (B))

Seeds of weeds velvetleaf, redroot pigweed, common ragweed, green foxtail and large crabgrass and seeds of corn were sown in 1/5,000-are Wagner pots filled with upland soil, and covered with upland soil. The seeds were grown in a greenhouse, and at the stage of 3˜4 leaves of these plants, a predetermined amount of the herbicide prepared in the above (1) was suspended in water, and the suspension was uniformly sprayed onto leaf and stalk portions at a rate of 2,000 liters/hectare. Then, the plants were grown in the greenhouse, and on the 30th day after the treatment, the herbicide was evaluated for herbicidal efficacy and phytotoxicity to the corn on the basis of the ratings shown in (2). Tables 8 and 9 show the results.

TABLE 8

| Com- | | | | | | | Phyto- |
|---|---|---|---|---|---|---|---|
| pound | Dosage | Herbicidal Efficacy | | | | | toxicity |
| No. | g/ha | AA | CC | DD | EE | FF | Corn |
| 1 | 80 | 5 | 5 | 5 | 5 | 5 | − |
| 2 | 80 | 5 | 5 | 5 | 5 | 5 | − |
| 3 | 80 | 5 | 5 | 5 | 5 | 5 | − |
| 4 | 80 | 5 | 5 | 5 | 5 | 5 | − |
| 5 | 80 | 5 | 5 | 5 | 5 | 5 | − |
| 6 | 80 | 5 | 5 | 5 | 5 | 5 | − |
| 7 | 80 | 5 | 5 | 5 | 5 | 5 | − |
| 8 | 80 | 5 | 5 | 5 | 5 | 5 | − |
| 12 | 80 | 5 | 5 | 5 | 5 | 5 | − |
| 13 | 80 | 5 | 5 | 5 | 5 | 5 | − |
| 14 | 80 | 5 | 5 | 5 | 5 | 5 | − |
| 15 | 80 | 5 | 5 | 5 | 5 | 5 | − |
| 18 | 80 | 5 | 5 | 5 | 5 | 5 | − |
| 19 | 80 | 5 | 5 | 5 | 5 | 5 | − |
| 21 | 80 | 5 | 5 | 5 | 5 | 5 | − |
| 24 | 80 | 5 | 5 | 5 | 5 | 5 | − |
| 25 | 80 | 5 | 5 | 5 | 5 | 5 | − |
| 26 | 80 | 5 | 5 | 5 | 5 | 5 | − |

Notes: AA = Velvetleaf, CC = Redroot Pigweed, DD = Common ragweed, EE = Green foxtail, FF = Large crabgrass

TABLE 9

| Com- | | | | | | | Phyto- |
|---|---|---|---|---|---|---|---|
| pound | Dosage | Herbicidal Efficacy | | | | | toxicity |
| No. | g/ha | AA | CC | DD | EE | FF | Corn |
| 27 | 80 | 5 | 5 | 5 | 5 | 5 | − |
| 29 | 80 | 5 | 5 | 5 | 5 | 5 | − |
| 30 | 80 | 5 | 5 | 5 | 5 | 5 | − |
| 37 | 80 | 5 | 5 | 5 | 5 | 5 | − |
| 39 | 80 | 5 | 5 | 5 | 5 | 5 | − |
| 42 | 80 | 5 | 5 | 5 | 5 | 5 | − |
| 49 | 80 | 5 | 5 | 5 | 5 | 5 | − |
| 50 | 80 | 5 | 5 | 5 | 5 | 5 | − |
| 51 | 80 | 5 | 5 | 5 | 5 | 5 | − |
| 52 | 80 | 5 | 5 | 5 | 5 | 5 | − |
| (A) | 80 | 2 | 0 | 2 | 1 | 1 | − |
| (B) | 80 | 2 | 2 | 5 | 4 | 5 | ++ |

Notes: AA = Velvetleaf, CC = Rough Pigweed, DD = Common ragweed, EE = Green foxtail, FF = Large crabgrass

The results in Tables 8 and 9 show that the herbicides of the present invention cause no phytotoxicity on corn and can selectively control a broad range of principal cropland weeds from monocotyledonous weeds to dicotyledonous weeds at a low dosage. On the other hand, it is shown that Compound (A) is inferior in herbicidal efficacy, particularly clearly poor in activity against redroot pigweed. It is also shown that Compound (B) is inferior in herbicidal efficacy against velvetleaf and redroot pigweed.

1. Pyrazole derivatives of the general formula (I), or salts thereof, 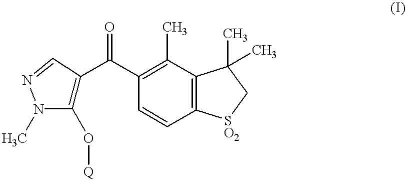
wherein Q is a hydrogen atom, a group of —SO₂—R¹, —CO—R¹ or —CH₂CO—R¹, in which R¹ is a C₁-C₈ alkyl group, a C₃-C₈ cycloalkyl group, a C₁-C₈ haloalkyl group or a group of the formula (II), 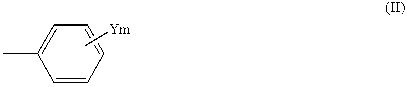
in which Y is a halogen atom, a nitro group, a C₁-C₄ alkyl group, a C₁-C₄ alkoxy group or a C₁-C₄ haloalkyl group, and m is an integer of 0 to 3, provided that when m is 2 or 3, each of Ys may be different or the same.

2. The pyrazole derivatives or salts thereof according to claim 1, wherein the salts are alkali metal salts or organic amine salts.

3. A herbicide containing, as an active ingredient, at least one selected from pyrazole derivatives of the general formula (I), or salts thereof, 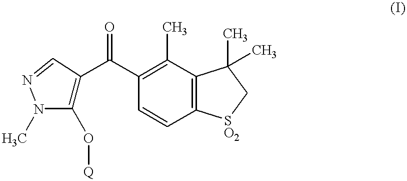
wherein Q is a hydrogen atom, a group of —SO₂—R¹, —CO—R¹ or —CH₂CO—R¹, in which R¹ is a C₁-C₈ alkyl group, a C₃-C₈ cycloalkyl group, a C₁-C₈ haloalkyl group or a group of the formula (II), 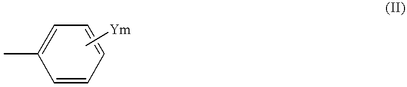
in which Y is a halogen atom, a nitro group, a C₁-C₄ alkyl group, a C₁-C₄ alkoxy group or a C₁-C₄ haloalkyl group, and m is an integer of 0 to 3, provided that when m is 2 or 3, each of Ys may be different or the same.

4. The herbicide according to claim 3, wherein the herbicide is used for controlling weeds in a corn field.

5. The herbicide according to claim 3, wherein the herbicide has a preparation form of a wettable powder, an emulsifiable concentrate, a dust or granules.
